(19) United States
(12) Patent Application Publication
(10) Pub. No.: US 2018/0325502 A1
(43) Pub. Date: Nov. 15, 2018

(54) MULTI-FACETED NEEDLE TIP AND METHOD OF MANUFACTURING

(71) Applicant: Devicor Medical Products, Inc., Cincinnati, OH (US)

(72) Inventor: Kinito Swader, Fairfield, OH (US)

(73) Assignee: Devicor Medical Products, Inc., Cincinnati, OH (US)

(21) Appl. No.: 15/755,840

(22) PCT Filed: Aug. 31, 2016
(86) PCT No.: PCT/US16/49637

§ 371 Date: Feb. 27, 2018

Related U.S. Application Data
(60) Provisional application No. 62/212,197, filed on Aug. 31, 2015.

(51) Int. Cl. A61B 10/02; A61B 17/34

(52) U.S. Cl. CPC A61B 10/0275; A61B 2017/00526; A61B 17/3421

(57) ABSTRACT

A needle for use with a biopsy device is described and claimed. The needle is manufactured using a metal injection molding or “MIM” process. The needle includes a piercing tip, wherein the piercing tip includes a base portion, a first cutting portion and a second cutting portion, wherein the first cutting portion and the second cutting portion extend distally from the base portion, wherein the base portion defines a first diameter, wherein the first diameter of the base portion corresponds to the first diameter of the cannula, wherein the first cutting portion defines a first cutting edge and a second cutting edge, wherein at least a portion the first cutting edge and at least a portion of the second cutting edge extends outwardly beyond the first diameter of the base portion.

### BACKGROUND OF THE INVENTION

Biopsy samples have been obtained in a variety of ways in various medical procedures including open and percutaneous methods using a variety of devices. For instance, some biopsy devices may be fully operable by a user using a single hand, and with a single insertion, to capture one or more biopsy samples from a patient. In addition, some biopsy devices may be tethered to a vacuum module and/or control module, such as for communication of fluids (e.g., pressurized air, saline, atmospheric air, vacuum, etc.), for communication of power, and/or for communication of commands and the like. Other biopsy devices may be fully or at least partially operable without being tethered or otherwise connected with another device. Biopsy devices may be used under stereotactic guidance, ultrasound guidance, Mill guidance, Positron Emission Mammography (“PEM” guidance), Breast-Specific Gamma Imaging (“BSGI”) guidance or otherwise.

The state of the art technology for conducting a breast biopsy is to use a vacuum-assisted breast biopsy device. A current textbook in this area is “Vacuum-Assisted Breast Biopsy with Mammotome®”, available Nov. 11, 2012, copyright 2013 by Devicor Medical Germany GmBh, published in Germany by Springer Medizin Verlag, Authors: Markus Hahn, Anne Tardivon and Jan Casselman, ISBN 978-3-642-34270-7, http://www.amazon.com/Vacuum-Assisted-Breast-Biopsy-Mammotome-Diagnostic/dp/3642342701?ie=UTF8&keywords=vacuum%20assisted%20breast%20 biopsy%20with%20Mammotome&qid=1460663723&ref_=sr_1_1&sr=8-1.

Known biopsy devices are disclosed in U.S. Pat. No. 5,526,822, entitled “Method and Apparatus for Automated Biopsy and Collection of Soft Tissue,” issued Jun. 18, 1996; U.S. Pat. No. 6,086,544, entitled “Control Apparatus for an Automated Surgical Biopsy Device,” issued Jul. 11, 2000; U.S. Pub. No. 2003/0109803, entitled “Mill Compatible Surgical Biopsy Device,” published Jun. 12, 2003; U.S. Pat. No. 7,507,210, entitled “Biopsy Cannula Adjustable Depth Stop,” issued Mar. 24, 2009; U.S. Pub. No. 2006/0074345, entitled “Biopsy Apparatus and Method,” published Apr. 6, 2006; U.S. Pub. No. 2007/0118048, entitled “Remote Thumbwheel for a Surgical Biopsy Device,” published May 24, 2007; U.S. Pub. No. 2008/0214955, entitled “Presentation of Biopsy Sample by Biopsy Device,” published Sep. 4, 2008; U.S. Pub. No. 2009/0171242, entitled “Clutch and Valving System for Tetherless Biopsy Device,” published Jul. 2, 2009; U.S. Pub. No. 2010/0152610, entitled “Hand Actuated Tetherless Biopsy Device with Pistol Grip,” published Jun. 17, 2010; U.S. Pub. No. 2010/0160819, entitled “Biopsy Device with Central Thumbwheel,” published Jun. 24, 2010; U.S. Pub. No. 2010/0317997, entitled “Tetherless Biopsy Device with Reusable Portion,” published Dec. 16, 2010; and U.S. Pat. No. 8,764,680, entitled “Handheld Biopsy Device with Needle Firing,” issued Jul. 1, 2014. The disclosure of each of the above-cited U.S. Patents, U.S. Patent Application Publications, and U.S. Non-Provisional Patent Applications is incorporated by reference herein.

Each of these known breast biopsy devices has a needle which is used to penetrate the tissue of the breast that the biopsy is being performed on. Currently known needle manufacturing techniques for surgical implements are used in manufacturing these needles.

U.S. Pat. No. 4,932,961 “Surgical Needle Configuration with Five-Sided Cross-Section” issued on Jun. 12, 1990. This patent describes and claims a needle having three fluted edges, all of the same angular size. The needle presents a five sided cross-section at a tapered end. This results in easier tissue penetration, reduced cross-sectional needle area, better wound opening area performance and minimized tissue distortion.

U.S. Pat. No. 5,403,344 “Multi-Faceted Surgical Needle” issued on Apr. 4, 1995. It describes and claims a surgical suturing needle with a tapered needle head with a multi-faceted cross-section. The cross-section is formed by three circumferentially-spaced cutting edges formed at a primary angle and a plurality of extended legs, each extending from one of the primary angles and formed at a secondary angle. In addition, a plurality of connecting surfaces adjoin adjacent extended legs.

Metal injection molding (MIM) is a metalworking process by which finely-powdered metal is mixed with a measured amount of binder material to comprise a “feedstock” capable of being handled by plastic processing equipment through a process known as injection mold forming. Dimensional tolerances of ±0.003 inches per linear inch can be commonly held, and far closer restrictions on tolerance are possible with expert knowledge of molding and sintering. MIM can produce parts where it is difficult, or even impossible, to efficiently manufacture an item through other means of fabrication. Increased costs for traditional manufacturing methods inherent to part complexity, such as internal/external threads, miniaturization, or brand identity marking, typically do not increase the cost in a MIM operation due to the flexibility of injection molding. https://en.wikipedia.org/wiki/Metal_injection_molding.

U.S. Published Patent Application US 2008/0281224 A1, “Biopsy Device Needle Tip” published on Nov. 13, 2008 and stands abandoned as of Sep. 16, 2011. This patent application described and claimed a biopsy device having a cannula with a distal tip. The distal tip includes a blade, and can be a unitary metal injection molded component including a base and a blade. The blade has a hardness of at least 40 HRC. The blade can be hardened, polished and then honed to provide a sharp leading edge.

U.S. Pat. No. 8,342,851B1, “Tissue Model for Testing Biopsy Needles” describes and claims a tissue model and test method. The tissue model can be used to simulate tissue during design and testing of biopsy needle tip configurations. For instance, the tissue model can be used to estimate the force required to penetrate natural breast tissue with a particular needle tip configuration. On col. 3, lines 8-13, it is stated, “The probe assembly 28 can include an elongate outer cannula 80 having a side tissue receiving port 86 and a distal tip 94. Distal tip 94 can be a metal injection molded (MIM) component which is attached, such as by welding, gluing, brazing, or other suitable joining methods to the distal end of outer cannula 80.”

Currently, certain breast biopsy devices manufactured and sold by Devicor Medical Products Inc. of Cincinnati, Ohio, have tri-point needles, where the needle tip itself is manufactured by MIM. With those products, the needle tip has to be welded to the rest of the parts making up the needle. With these probes, the welding is an additional step in the production of the needle and welding can lead to complications concerning the straightness and deformation of the finished product.

It would be desirable to replace existing processes to manufacture needles for breast biopsy devices with processes that have fewer steps and produce a needle with the same functionality or a needle with an improved functionality.

While several systems and methods have been made and used for obtaining a biopsy sample, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements. In the drawings some components or portions of components are shown in phantom as depicted by broken lines.

FIG. 1 depicts a partial perspective exploded view of a needle for use with a breast biopsy device; this needle is PRIOR ART and is NOT considered an example of the instant claimed invention;

FIG. 2 depicts the step by step current process to manufacture the needle of FIG. 1. This process is PRIOR ART and is NOT considered an example of the instant claimed invention;

FIG. 3 depicts a partial perspective view of an exemplary alternative needle that may be incorporated into a breast biopsy device;

FIG. 4 depicts a partial exploded view of the needle of FIG. 3;

FIG. 5 depicts a front cross-sectional view of the needle of FIG. 3, with the cross-section taken along line 5-5 of FIG. 4;

FIG. 6 depicts the step by step Metal Injection Molding (MIM) process to manufacture a needle for a breast biopsy device. This process is an example of one embodiment of the instant claimed invention;

FIG. 7 depicts the manufacturing process of the instant claimed invention relative to the prior art manufacturing process as shown in FIG. 2;

FIG. 8A depicts another partial perspective view of the needle of FIG. 1;

FIG. 8B depicts a perspective view of an exemplary alternative piercing tip with four facets that may be readily incorporated into the needle of FIG. 3;

FIG. 8C depicts a front elevational view of the piercing tip of FIG. 8B;

FIG. 9A depicts a perspective view of an exemplary alternative piercing tip with five facets that may be readily incorporated into the needle of FIG. 3;

FIG. 9B depicts another perspective view of the piercing tip of FIG. 9A;

FIG. 9C depicts a perspective of an exemplary alternative piercing tip with nine facets that may be readily incorporated into the needle of FIG. 3; and

FIG. 9D depicts another perspective view of the needle of FIG. 5C.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### SUMMARY OF THE INVENTION

The first aspect of the instant claimed invention is a needle for use with a biopsy device, wherein the needle comprises: an elongate cannula extending distally from a body of the biopsy device, wherein the elongate needle defines a first diameter and a cutout portion; and an insert member, wherein the insert member is insertable into the cutout portion of the cannula, wherein the insert member comprises: a tube portion, wherein the tube portion defines a lateral aperture; and a piercing tip, wherein the piercing tip includes a base portion, a first cutting portion and a second cutting portion, wherein the first cutting portion and the second cutting portion extend distally from the base portion, wherein the base portion defines a first diameter, wherein the first diameter of the base portion corresponds to the first diameter of the cannula, wherein the first cutting portion defines a first cutting edge and a second cutting edge, wherein at least a portion the first cutting edge and at least a portion of the second cutting edge extends outwardly beyond the first diameter of the base portion.

The second aspect of the instant claimed invention is an insert member for use in a needle of a biopsy device, wherein the needle includes a cutout portion, wherein the insert member is fixedly secured within the cutout portion, wherein the insert member comprises: a tubular portion, and a piercing tip, wherein the tubular portion extends proximally from the piercing tip, wherein the piercing tip comprises: a base portion, wherein the base portion is adjacent to the tubular portion, wherein the base portion defines a longitudinal diameter and a transverse diameter, a first cutting portion, wherein the first cutting portion includes a first cutting edge and a second cutting edge, wherein the first cutting edge and the second cutting edge is aligned with the longitudinal diameter of the base portion, and a second cutting portion, wherein the second cutting portion defines a first cutting edge and a second cutting edge, wherein the first cutting edge and the second cutting edge are aligned with the transverse diameter of the base portion.

The third aspect of the instant claimed invention is a piercing tip for use in a needle of a biopsy device, the piercing tip comprising: a base portion; a first cutting portion, wherein the first cutting portion defines a first cutting edge, wherein the first cutting edge defines a cut length that is greater than a diameter defined by the base portion; and a second cutting portion, wherein the second cutting portion defines a plurality of facets, wherein each facet intersects with another facet to form a second cutting edge, wherein the second cutting edge is disposed proximally relative to the first cutting edge of the first cutting portion.

The fourth aspect of the instant claimed invention is a single piece metal injection molded needle tip and cutter shelf for the needle assembly of a biopsy device.

### DETAILED DESCRIPTION OF THE INVENTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

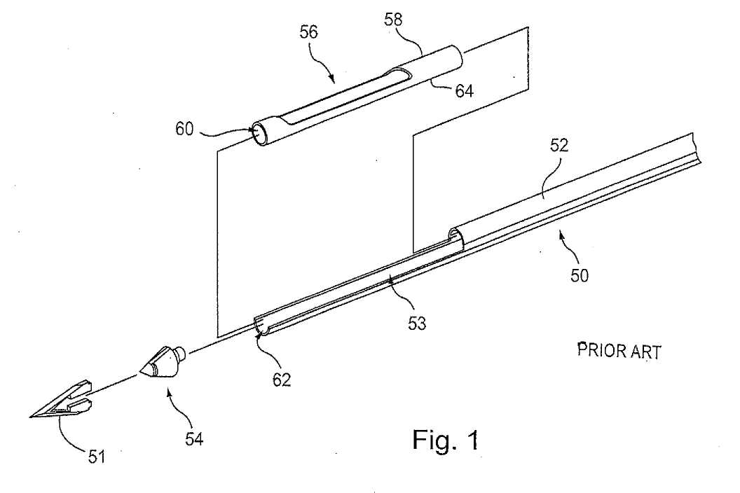

FIG. 1 shows PRIOR ART current needle (50) that may be readily incorporated into a biopsy device (not shown) for breast biopsy procedures. Current needle (50) is prior art. Current needle (50) includes a cannula (52), a piercing tip (54), and a lateral aperture (56) located proximal to piercing tip (54). Piercing tip (54) is configured to pierce and penetrate tissue, without requiring a high amount of force. Typically in biopsy procedures a small nick in the skin is made with a scalpel prior to inserting the tissue piercing tip of the biopsy device. It is anticipated that this small nick in the skin would still need to be made using piercing tip (54) although it is possible that in working with some patients, piercing tip (54) might be functional enough to be used without requiring an opening to be pre-formed in the tissue prior to insertion of piercing tip (54).

Alternatively, piercing tip (54) may be blunt (e.g., rounded, flat, etc.) if desired.

Piercing tip (54) may also be configured to provide greater echogenicity than other portions of needle (50), providing enhanced visibility of tip (54) under ultrasound imaging. By way of example only, piercing tip (54) may be configured in accordance with any of the teachings in U.S. Pat. Pub. No. 2012/0059247, entitled “Echogenic Needle for Biopsy Device,” published Mar. 8, 2012, the disclosure of which is incorporated by reference herein. Other suitable configurations that may be used for piercing tip (54) will be apparent to those of ordinary skill in the art in view of the teachings herein.

Lateral aperture (56) is sized to receive prolapsed tissue during operation of the biopsy device. Although not shown, it should be understood that a hollow tubular cutter having a sharp distal edge is located within a first lumen (60) of current needle (50). The cutter is operable to rotate and translate relative to current needle (50) and past lateral aperture (56) to sever a tissue sample from tissue protruding through lateral aperture (56). For instance, the cutter may be moved from an extended position to a retracted position where the distal end of the cutter is just proximal of the proximal end of the lateral aperture (56), thereby “opening” lateral aperture (56) to allow tissue to protrude there-through; then from the retracted position back to the extended position to sever the protruding tissue. Mechanical components in the biopsy device such actuation of the cutter, as described in any reference cited herein or otherwise. Other suitable alternative versions, features, components, configurations, and functionalities for providing cutter actuation will be apparent to those of ordinary skill in the art in view of the teachings herein.

In some instances, current needle (50) may be manually rotated to orient lateral aperture (56) at any desired angular position about the longitudinal axis of needle (50). Suitable structures and/or features for rotation of current needle (50) may be constructed and operable in accordance with the teachings of U.S. Pat. No. 8,764,680 and/or in any other suitable fashion. Various other suitable ways in which manual rotation of needle (50) may be provided will be apparent to those of ordinary skill in the art in view of the teachings herein. It should also be understood that rotation of current needle (50) may be automated in various ways, including but not limited to the various forms of automatic or mechanized needle rotation described in various references that are cited herein.

Current needle (50) has a non-circular cross-sectional shape such as, a generally ovular shape defined by cannula (52). In the present example, longitudinal wall (64) is formed by an internal tube (58) which also forms lateral aperture (56). Tube (58) is inserted into a cut out portion (53) in cannula (52). Tube (58) may be fixed to cannula (52) by any suitable means such as laser welding, adhesive bonding, or the like.

Current needle (50) includes a longitudinal wall (64) that is formed when tube (58) is inserted into cut out portion (53). Longitudinal wall (64) extends proximally from the proximal portion of tip (54). While wall (64) does not extend along the full length of current needle (50) in this example, it should be understood that wall (64) may extend the full length of current needle (50) if desired.

Wall (64) defines a second lumen (62) that is lateral to and parallel to the cutter.

Although not shown, it should be understood that wall (64) includes a plurality of openings that provide fluid communication between second lumen (62) and first lumen (60), as well as fluid communication between second lumen (62) and the lumen (not shown) of the cutter. For instance, second lumen (62) may selectively provide atmospheric air to vent the lumen of the cutter during operation of the biopsy device. The openings are arranged such that at least one opening is located at a longitudinal position that is distal to the distal edge of lateral aperture (56). Thus, the lumen of the cutter and second lumen (62) may remain in fluid communication even when the cutter is advanced to a position where the distal cutting edge of the cutter is located at a longitudinal position that is distal to the longitudinal position of the distal edge of lateral aperture (56). Of course, as with any other component described herein, any other suitable configurations may be used.

As can also be seen in FIG. 1, piercing tip (54) of the present example comprises a multi-member tip assembly. In particular, piercing tip (54) comprises a blade (51) and a coupling member (57). Blade (51) generally comprises a flat planer portion with a pair of sharpened edges oriented distally to form a sharp distal tip. Coupling member (57) comprises a generally conical member that is configured to receive blade (51). When assembled, at least a portion of blade (51) is received within coupling member (57) and securely fastened thereto. Coupling member (57) is then received into the distal end of cannula (52) and the distal end of tube (58). Although not shown, it should be understood that in some examples coupling member (57) may comprise multiple parts that fasten together to secure blade (51) thereto. By way of example only, blade (51) and coupling member (57) may be constructed in accordance with the teachings of U.S. Pat. No. 8,801,742, the disclosure of which is incorporated by reference herein.

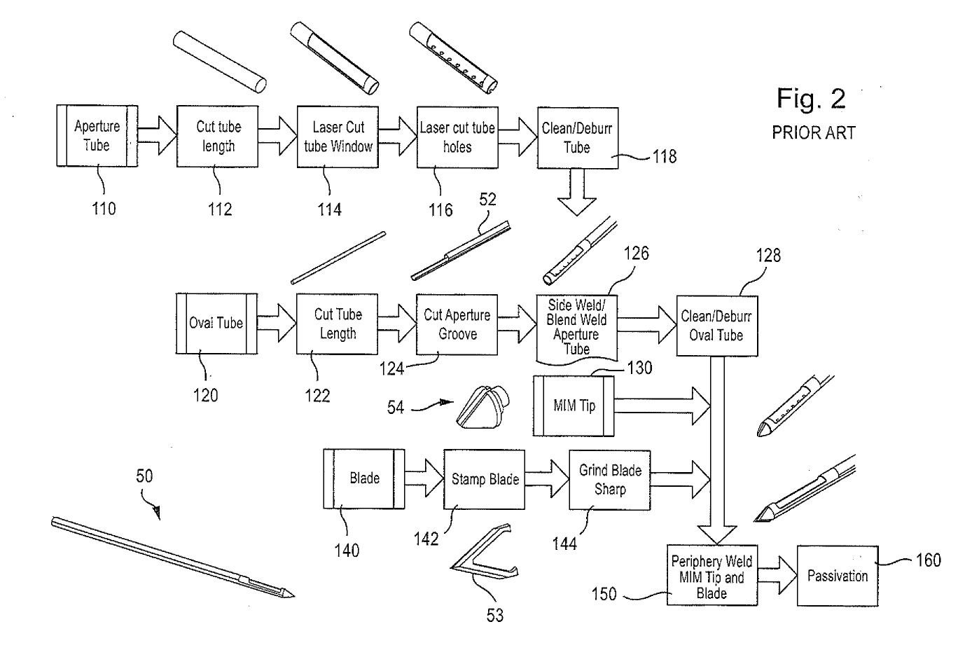

FIG. 2 depicts the current method of manufacture for current needle (50) used in current biopsy devices. The Method shown in FIG. 2 is PRIOR ART and is NOT an example of the instant claimed invention. In this method, internal tube (58), cannula (52), piercing tip (54), and blade (51) are individually assembled separately and then combined together. For instance, in blocks (110-118), internal tube (58) is constructed though a series of manufacturing steps. In bock (110), an elongate tube is provided. The elongate tube is then cut to a predetermined length in block (112). Once cut to a desired length, a window for lateral aperture (56) is cut into the elongate tube that has been cut to length in block (114). With the window for lateral aperture (56) cut into the elongate tube that has been cut to length, holes can be laser cut into the tube below lateral aperture (56) as represented by block (116). Once the process of cutting holes in tube is complete, the tube is cleaned and deburred in block (118) and internal tube (58) is complete.

In blocks (120-124) cannula (52) is constructed. Once constructed as desired, cannula (52) can then be combined with internal tube (58) as represented by blocks (126, 128). To prepare cannula (52), an elongate oval shaped tube is first provided as represented by block (120). The tube is then cut to a predetermined length as indicated by block (122). A cutting operation such as laser cutting then cuts a groove in the distal end of the tube as represented by block (124).

At this stage, cannula (52) has been prepared and is in a condition to be joined with internal tube (58) using the steps as represented by blocks (126, 128). To join internal tube (58) with cannula (52), internal tube (58) is first positioned within the groove formed during the step represented by block (124). Internal tube (58) is then welded into position on cannula (52) as represented by block (126). Welding may be performed using any suitable welding process such as laser welding, electron beam welding, gas tungsten arc welding, and/or etc. For a list of known welding processes please see https://en.wikipedia.org/wiki/List_of_welding_processes.

Once internal tube (58) is joined to cannula (52), the combined assembly of internal tube (58) and cannula (52) is subjected to a cleaning and deburring process as represented by block (128).

Piercing tip (54) is prepared by a metal injection molding process (MIM). This process is represented by block (130). In the MIM process, a feedstock comprising fine grained metal powder and binding agents is injected as a liquid into a mold configured to form the generally conical shape of piercing tip (54). This forms a near-net-shape workpiece that is cooled and demolded. Once demolded, the workpiece is subjected to various treatment methods using solvents and/or thermal processes to extract binding materials. The resulting workpiece is fragile and porous at this stage. The workpiece is then subjected to a sintering process that condenses the remaining metal to harden and remove porosity from the structure of the workpiece. Once sintering is complete, the workpiece is subjected to grinding operations to achieve desired tolerances, thereby providing the final version of piercing tip (54).

Blade (51) is prepared using the steps as represented by blocks (140-144). In particular, a piece of sheet metal having a predetermined thickness is provided as represented by block (140). The sheet metal is then subjected to a stamping process as represented by block (142). The stamping process generates a blank part by separating a portion of the sheet metal having the general shape of blade (51) from the rest of the sheet metal. This blank part is then subjected to a grinding process as represented by block (144). This grinding process creates the sharp edges on the distal end of blade (51). Once grinding is complete, blade (51) is prepared and is ready for combination with other elements of the current needle.

Once the various parts of needle have been prepared, they are typically combined as represented by block (150). In particular, the assembly of internal tube (58) and cannula (52) combined as represented by block (126) is joined with piercing tip (54) and blade (51). As represented by block (150), blade (51) is inserted onto piercing tip (54). The combined assembly of blade (51) and piercing tip (54) is then inserted into the distal end formed by cannula (52) and internal tube (58). The peripheral edges are then welded to join piercing tip (54) to the assembly of cannula (52) and internal tube (58), and blade (51) to piercing tip (54). Any suitable welding process, as previously described herein, can be used at this stage as similarly described above with respect to block (126).

Once cannula (52), internal tube (58), piercing tip (54), and blade (51) are joined, the entire assembly is passivated as represented by block (160). In the present example, passivation is used to maximize the corrosion resistance of current needle (50). This step generally involves subjecting needle to a passivating acid bath. Suitable passivating techniques may be varied depending on the material used and the particular amount of passivation that may be desired. Suitable passivation methods will be apparent to those of ordinary skill in the art in view of the teachings herein.

In reviewing the method of FIG. 2 it is observable that alternate methods of manufacturing would be desirable. One possible alternative method is to combine piercing tip (54) and tube (58) into a single discrete unit. Such a combination may be desirable to improve ease of manufacturability, reduce cost, and/or strengthen current needle (50). Additionally, it may be desirable to provide various alternative piercing tip (54) geometries to promote ease of penetration of needle though tissue.

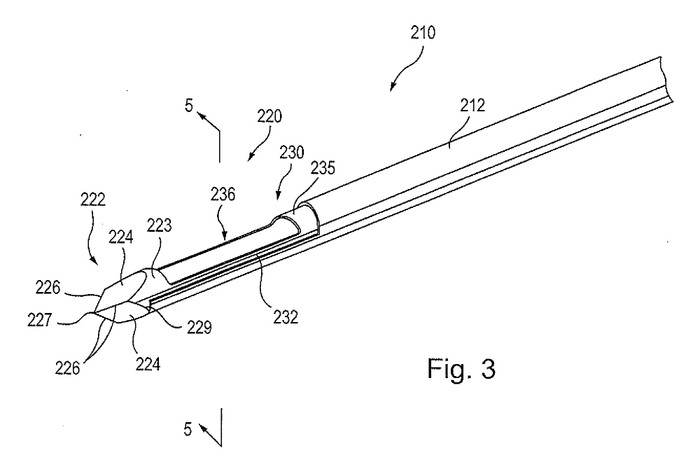

FIGS. 3-6 show an example of needle (210) that may be readily incorporated into the biopsy device in lieu of the current needle described above. Needle (210) is an example of the instant claimed invention. Needle (210) has certain elements in common with current needle (50) described above, except where as otherwise noted herein. For instance, like with current needle (50), needle (210) comprises a cannula (212), a piercing tip (227), and a lateral aperture (236) located proximal to tip (227). Cannula (212) is substantially the same as cannula (52) described above. For instance, cannula (212) extends distally from a hub member (not shown) and comprises a generally oval-shaped cross-section. Also like cannula (52), cannula (212) comprises a cut-out portion (214) for receiving various components of needle (210) as will be described in greater detail below.

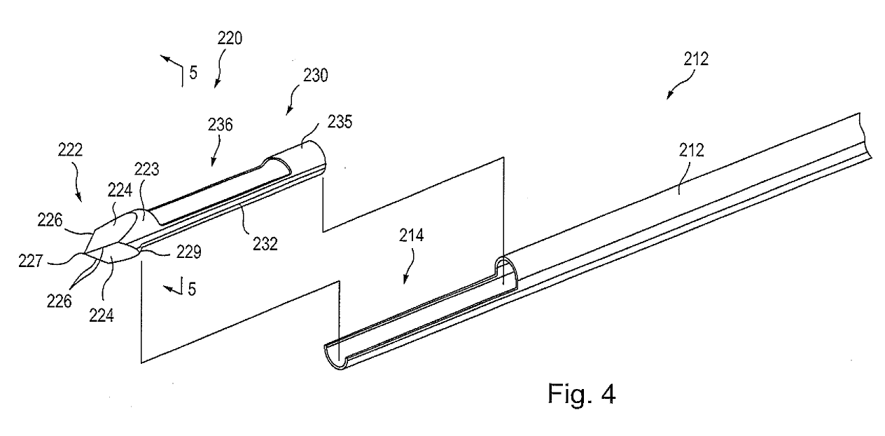

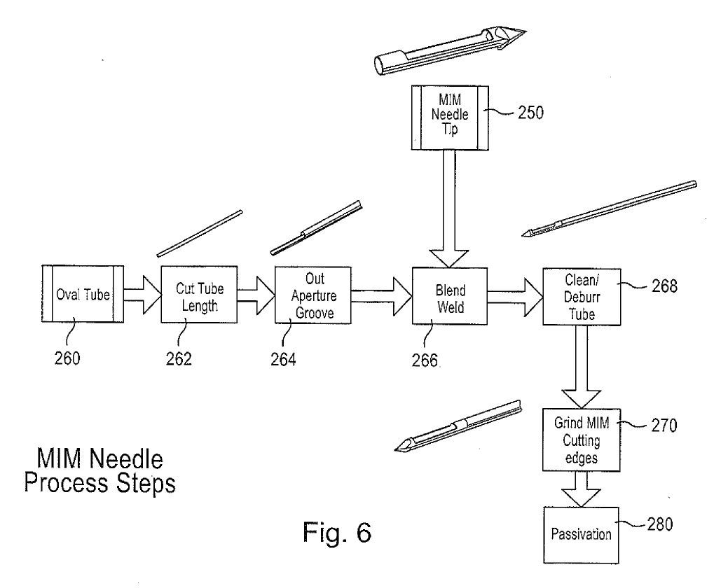

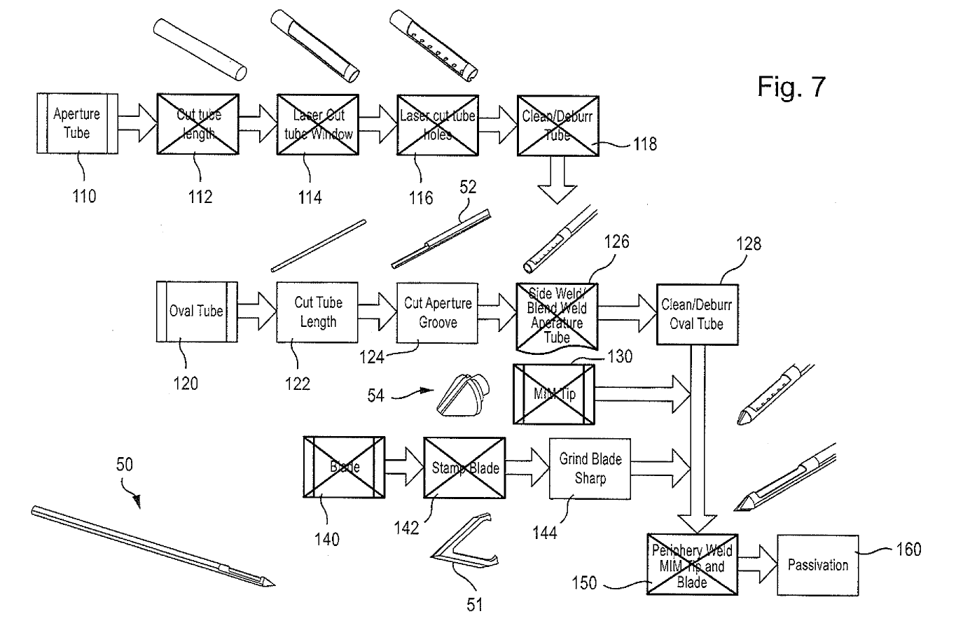

In contrast to current needle (50) described above, needle (210) of the present example includes an insert member (220), which is similar to a combination of piercing tip (54), blade (51), and tube (58) of current needle (50). In particular, insert member (220) is insertable into cut-out portion (214) of cannula (212) to provide piercing tip (227), and lateral aperture (236). As can best be seen in FIG. 4, insert member (220) comprises a tip portion (222) and a tube portion (230). As will become apparent in later discussions, it is important to note that tip portion (222) and tube portion (230) do not have to be welded together to form insert member (220), but rather are metal injection molded together during the process shown in FIG. 6.

As can best be seen in FIG. 4, tip portion (222) comprises a generally solid body (223).

Body (223) has a generally ovular transverse cross-sectional shape, which corresponds to the ovular shape of cannula (212). A plurality of tip faces (224) circumscribe the distal end of body (223). Each tip face (224) intersects with another face (224) or faces (224) to form cutting edges (226). In the present example, tip portion (222) comprises three tip faces (224), although any suitable number of tip faces (224) may be used. Cutting edges (226) converge to form piercing tip (227). In other examples, not all cutting edges (226) converge to form piercing tip (227). For instance, in some examples cutting edges (226) intersect with an integral cutting blade (not shown) extending outwardly and longitudinally along each side of tip portion (222). Such a cutting blade may converge upon itself to form piercing tip (227).

In contrast to the prior art method shown in FIG. 2, in FIG. 6 the method of manufacturing the needle of the instant claimed invention shows the MIM needle tip is completely formed and then welded to the oval tube forming cannula (212) with needle tip in place. In FIG. 2, block (150) shows one of the last steps is the periphery weld of MIM tip and blade.

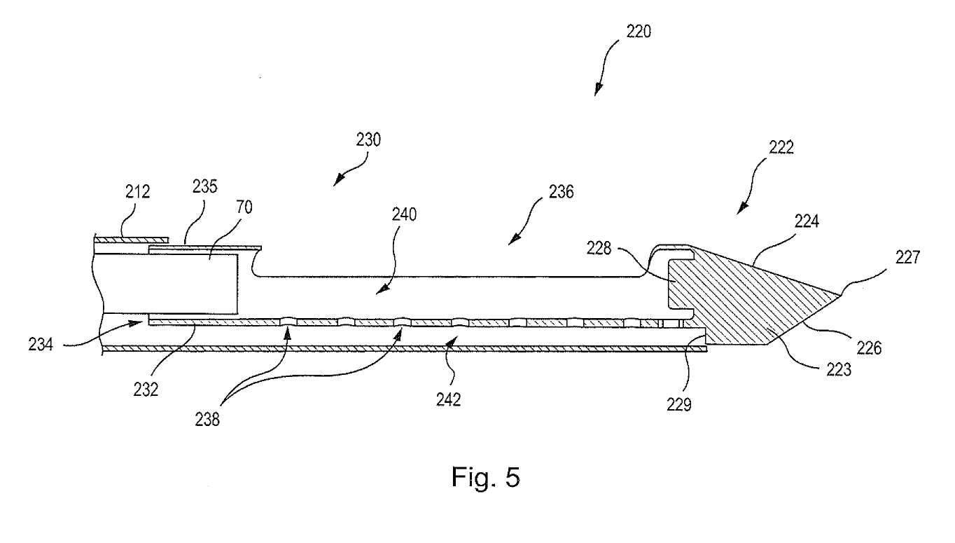

As can be seen in FIG. 5, the proximal end of body (223) defines a longitudinal protrusion (228) and a lateral protrusion (229). Longitudinal protrusion (228) extends distally from the proximal end of body (223) into tube portion (230). As will be described in greater detail below, longitudinal protrusion (228) is configured to receive cutter (70) of biopsy device (10) to more readily sever tissue samples.

Lateral protrusion (229) is defined by the interface between body (223) and tube portion (230). As will be described in greater detail below, tube portion (230) defines a generally circular cross-section. This generally circular cross-section extending proximally from the generally ovular cross-section of body (223) defines lateral protrusion (229). Lateral protrusion (229) generally comprises a crescent shape. This crescent shape is configured to abut the distal end of cannula (212). Thus, as will be described in greater detail below, when inert member (220) is inserted into cut-out portion (214) of cannula (212), lateral protrusion (229) serves to block and seal at least a portion of cannula (212).

Tube portion (230) is shown as extending proximally from body (223). Tube portion (230) comprises an elongate cylindrical tube body (232) extending from the proximal end of tip portion (222), terminating in an open proximal end (234). Tube body (232) further comprises lateral aperture (236), which is cut into an upper portion of tube body (232). Lateral aperture (236) of the present example is substantially similar to lateral aperture (56) described above such that lateral aperture (236) is sized to receive prolapsed tissue during operation of biopsy device (10).

As can be seen in FIG. 5, tube body (232) further includes a plurality of apertures (238) arranged in a linear array. Apertures (238) are positioned within tube body (232) on a side of tube body (232) opposite of lateral aperture (236). As will be described in greater detail below, apertures (238) are configured to provide fluid communication to assist with transport of tissue samples through cutter (70).

Open proximal end (234) is positioned adjacent to a closed portion (235) of tube body (232). Closed portion (235) is generally formed as a solid portion of tube body (232) that is proximal to lateral aperture (236). The longitudinal length of closed portion (235) is shown as being shorter than the length of lateral aperture (236). In other examples, the longitudinal length of closed portion (235) is longer than lateral aperture (236), yet shorter than the length of cannula (212). As will be described in greater detail below, closed portion (235) is generally configured to abut the proximal end of cut-out portion (214) of cannula (212). As will also be described in greater detail below, closed portion (235) is sized to receive cutter (70) of the biopsy device such that closed portion (235) circumferentially surrounds the outer diameter of cutter (70), thereby maintaining a coaxial relationship between insert member (220) and cuter (70).

A cross-sectional view of needle (210), manufactured using the process shown in FIG. 6, is shown in FIG. 5. As can be seen, when needle (210) is in the assembled condition, insert member (220) is disposed within cut-out portion (214) of cannula (212) to define two lumens (240, 242). In particular, a cuter lumen (240) is defined by tube portion (230) of insert member (220). Cutter lumen (240) is generally configured to slidably receive cutter (70) through the cutting stroke of cutter (70). Thus, closed portion (235) of tube body (232) is sized to contain cutter (70) therein, even when cutter is in a retracted position.

A second, lateral lumen (242) is defined below cutter lumen (240). In particular, lateral lumen (242) is defined by at least a portion of tube body (232) and at least a portion of an inner wall of cannula (212). Because inert member (220) does not extend proximally for the full length of cannula (212), it should be understood that at least a portion of lateral lumen is also defined by cutter (70) and the inner wall of cannula (212). Thus, lateral lumen may extend the entire length of cannula (212) even though insert member (220) extends for a portion of cannula (212).

Apertures (238) in tube body (232) are disposed between cutter lumen (240) and lateral lumen (242). This positioning permits fluid communication between cutter lumen (240) and lateral lumen (242) via apertures (238). Additionally, because apertures (238) are positioned opposite to lateral aperture (236), it should be understood that such fluid location is localized to an area approximately adjacent to lateral aperture (236). In some examples, this arrangement of cutter lumen (240), lateral lumen (242), apertures (238), and lateral aperture (236) permits lateral lumen (242) to selectively communicate atmosphere to cutter lumen (240). In some examples the presence of atmosphere and selective times during the biopsy procedure may aid in advancing a severed tissue sample proximally though cutter. In other examples lateral lumen (242) may, in addition or in alternative to atmosphere, selectively supply vacuum and/or saline to cutter lumen to further assist with the tissue collection process and/or biopsy procedure generally.

To assemble needle (210), an operator uses the method of construction shown in FIG. 6. Initially the procedure begins with insert member (220) separated from cannula (212), as similarly shown in FIG. 4. At this stage, insert member (220) is produced by a MIM process as represented by block (250). As similarly described above with respect to piercing tip (54), the MIM process uses a granular metal powder that is mixed with a plastic and/or wax binder to produce a liquid feedstock. Next the feedstock is fed into a convention injection molding machine and the feedstock is injected into a mold having the shape of insert member (220). After molding, a post-molding insert member (220) is produced. Because of the presence of binders and/or wax, the post-molding insert member (220) is generally in a state requiring further processing. To remove binders and/or wax, the post-molding insert member (220) is subjected to a chemical or thermal treatment process. Finally, the part is subjected to a sintering process to place insert member (220) in a condition suitable for assembly into needle (210). Once sintering is complete, insert member (220) can be optionally subjected to grinding, cleaning, or other suitable finishing processes to bring insert member (220) within predetermined final tolerances.

It should be understood that in other examples insert member (220) may be formed by a variety of other processes. For instance, insert member (220) may be formed using conventional machining methods, and/or casting methods. In still other examples, insert member (220) may be formed by a metal 3D printing process. In yet other examples, insert member (220) may be formed using any other suitable process as will be apparent to those of ordinary skill in the art in view of the teachings herein.

Independently of producing insert member (220), cannula (212) may also be prepared as represented by blocks (260-264). At this stage cannula (212) is initially cut to a predetermined length from tube stock having the desired thickness of tube as represented by block (260). Next, cannula (212) is subjected to a cutting process (e.g., laser cutting) to include cut-out portion (214) as represented by block (264).

Once cut-out portion (214) is formed in cannula (212), an operator may begin assembly by inserting insert member (220) into cut-out portion (214). It should be understood that during insertion of insert member (220) at least a portion of insert member (220) may be inserted into cannula (212). For instance, the proximal end of tube portion (230) may be inserted into the distal end of cut-out portion (214). Similarly, lateral protrusion (229) of tip portion (222) may be inserted into the distal end of cannula (212). In alternative to the above, one or both of the proximal end of tube portion (230) or lateral protrusion (229) may merely abut cut-out portion (214) or cannula (212), respectively.

Once insert member (220) is positioned into cut-out portion (214), insert member (220) is secured to cannula (212) as represented by block (266). In one embodiment of the instant claimed invention, insert member (220) is secured to cannula (212) by laser welding, or any other suitable welding process, at the interface between insert member (220) and cannula (212). This process fluidly seals the interface between insert member (220) and cannula (212) such that the only accessible opening is lateral aperture (236). In other embodiments of the instant claimed invention, numerous other methods that secure and seal the interface between insert member (220) and cannula (212) may be used. For instance, in some examples the interface between cannula (212) and insert member (220) is secured and sealed using other welding process such as gas metal arc welding, gas tungsten arc welding, electron beam welding, or ultrasonic welding or other welding processes known to people of ordinary skill in the art of welding, see https://en.wikipedia.org/wiki/List_of_welding_processes.

In still other examples, non-welding methods may be used for securing and sealing such as adhesive bonding, diffusion bonding, or forging. Of course, any other suitable boding method may be used as will be apparent to those of ordinary skill in the art in view of the teachings herein.

Once joining of cannula (212) and insert member (220) is complete, needle (210) is cleaned and or deburred as represented by block (268). Once cleaned and deburred, the edges of insert member (220) are ground to their final tolerances as represented by block (270). As described above, grinding may alternatively be completed after the MIM process as represented by block (250). Thus, if grinding is performed in connection with the MIM process, further grinding as represented by block (270) may not be necessary. Regardless of when grinding is performed, it should be understood that grinding is typically necessary to provide a sufficiently sharp piercing tip (227) on insert member (220). This is due to limitations in the MIM process that result in a workpiece that is only near-net-shape.

After grinding is completed, needle (210) is subjected to passivation as represented by block (280). In the present example, passivation is used to maximize the corrosion resistance of needle (210). This step generally involves subjecting needle (210) to a passivating acid bath. Suitable passivating techniques may be varied depending on the material used and the particular amount of passivation that may be desired. Suitable passivation methods will be apparent to those of ordinary skill in the art in view of the teachings herein.

FIG. 6 compares the process for producing current needle (50) described above with the process for producing needle (210). As can be seen, the process for producing needle (210) results in elimination of as many as 6 process steps. This is due to the configuration of insert member (220) that generally combines internal tube (58), piercing tip (54), and blade (51) of needle (50) into a single part (e.g., insert member (220)), with that single part being formed by metal injection molding. This also results in the elimination of as many as two parts from needle (210) relative to current needle (50). Accordingly, it should be understood that the configuration of needle (210) results in enhanced manufacturing efficiencies relative to the configuration of current needle (50).

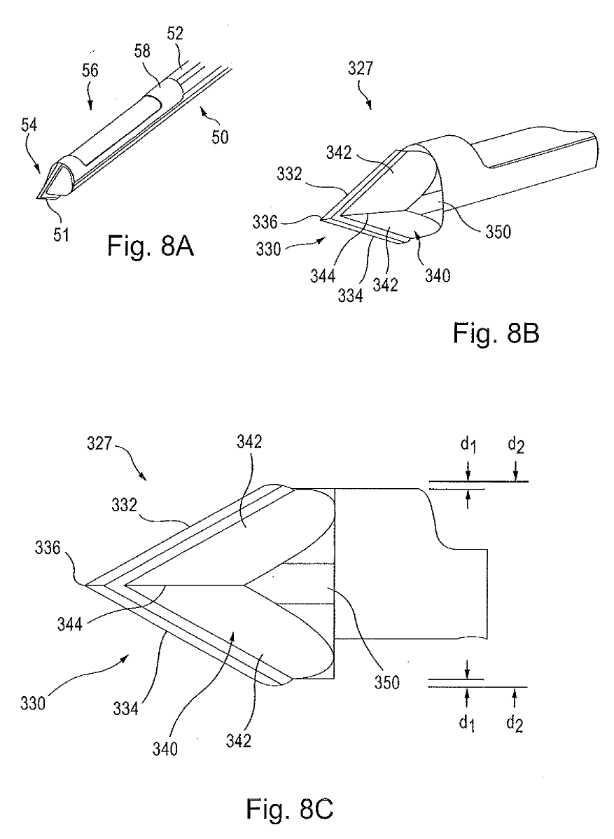

FIGS. 8B and 8C show an exemplary alternative piercing tip (327) that can be readily incorporated into inert member (220) described above. Piercing tip (327) is generally configured similarly to piercing tip described above (54), except piercing tip (327) is configured to include features to enhance the ease by which piercing tip (327) can penetrate tissue (e.g., force to penetrate). As will be described in greater detail below, various features of piercing tip (327) are configured to enhance force to penetrate by increasing the cut length of piercing tip (327). Additionally, features are included to reduce drag forces imparted onto needle (210) as it penetrates through tissue.

Piercing tip (327) comprises a first cutting portion (330), a second cutting portion (340), and a base portion (350). In the present example first cutting portion (330) is oriented distally of second cutting portion (340). Also in the present example, base portion (350) is generally disposed proximally of both first cutting portion (330) and second cutting portion (340).

Base portion (350) defines a shape that generally corresponds to the shape of cannula (212), described above. Thus, it should be understood that when attached to cannula (212), base portion (350) and cannula (212) form a flush interface such that there is a smooth transition between piercing tip (327) and cannula (212). Like with cannula (212), base portion (350) defines an oval-shaped cross-sectional shape. Thus, base portion (350) defines a longitudinal diameter (oriented vertically in FIGS. 8B and 8C) and a transverse diameter (oriented horizontally in FIGS. 8B and 8C). As will be described in greater detail below, first cutting portion (330) and second cutting portion (340) are both generally oriented relative the longitudinal and transverse diameters that are defined by base portion (350).

First cutting portion (330) defines a first longitudinal cutting edge (332) and a second longitudinal cutting edge (334). First longitudinal cutting edge (332) and second longitudinal cutting edge (334) each converge to form a sharp point (336). In addition, first longitudinal cutting edge (332) and second longitudinal cutting edge (334) are oriented along a plane that is parallel relative to the longitudinal diameter defined by base portion (350). As first longitudinal cutting edge (332) and second longitudinal cutting edge (334) extend proximally from sharp point (336), at least a portion of first longitudinal cutting edge (332) and second longitudinal cutting edge (334) also extend outwardly from the outer perimeter of base portion (350). As shown in FIG. 8C, this outward extension defines an outward extension distance (d1). Outward extension distance (d1) is configured to provide additional cutting of tissue beyond the longitudinal diameter of base portion (350). As will be described in greater detail below, this additional cutting relieves stress in tissue as it is being pierced to thereby reduce drag on needle (210).

First longitudinal cutting edge (332) and second longitudinal cutting edge (334) both extend relative to sharp point (336) at an angle to thereby define an axial extension leg and an outward extension leg. The sum of the outward extension leg of both first longitudinal cutting edge (332) and second longitudinal cutting edge (334) defines a cut length (d2) for first cutting portion (330). Cut length (d2) for first cutting portion (330) is summed with a cut length defined by second cutting portion (340) to define a total cut length for piercing tip (327). As will be described in greater detail below, the total cut length for piercing tip (327) is greater relative to the cut length of piercing tip (54). As will also be described in greater detail below, this generally results in a direct reduction in force to penetrate with piercing tip (327) relative to piercing tip (54).

Second cutting portion (340) is defined in base portion (350) by a plurality of faceted surfaces (342). Although only two faceted surfaces (342) are shown in FIGS. 8B and 8C, it should be understood that the present example includes four faceted surfaces (342), with two facets disposed on opposite of the faceted surfaces (342) shown in FIGS. 8B and 8C. Faceted surfaces (342) are shown in the present example as being disposed proximally relative to first cutting portion (330) such that second cutting portion (340) is generally proximally offset relative to first cutting portion (330). It should be understood that in other examples, second cutting portion (340) can be configured to be in any suitable position relative to first cutting portion (330). For instance, in some examples, the configuration can be reversed such that second cutting portion (340) is disposed distally of first cutting portion (330). Similarly, in other examples, first cutting portion (330) and second cutting portion (340) can be aligned such that there is no axial offset between the two.

Each faceted surface (342) meets with an adjacent faceted surface (342) to define a first lateral cutting edge (344) and a second lateral cutting edge (not shown). First lateral cutting edge (344) extends proximally and outwardly in parallel with a plane defined by the lateral diameter of base portion (350). Thus, first lateral cutting edge (344) is oriented perpendicularly relative to first longitudinal cutting edge (332) and second longitudinal cutting edge (334). Although not shown, it should be understood that the second lateral cutting edge is also extends proximally and outwardly in parallel with a plane defined by the lateral diameter of base portion (350). Thus, the second lateral cutting edge is also oriented perpendicularly relative to first longitudinal cutting edge (332) and second longitudinal cutting edge (334).

Like with first longitudinal cutting edge (332) and second longitudinal cutting edge (334) described above, first lateral cutting edge (344) and the second lateral cutting edge both define an axial extension leg and an outward extension leg. The sum of the outward extension leg for both first lateral cutting edge (344) and the second lateral cutting edge likewise defines a cut length (not shown) for second cutting portion (340). The sum of cut length (d2) for first cutting portion (330) and the cut length for second cutting portion (340) defines a total cut length for piercing tip (327).

A comparison of piercing tip (54) and piercing tip (327) can be seen in FIGS. 8A and 8B. As can be seen, piercing tip (327) defines a total cut length that is approximately twice that of the total cut length of piercing tip (54). This is generally the consequence of the presence of second cutting portion (340) that is present in piercing tip (327), but not present in piercing tip (54). It should be understood that the greater cut length present in piercing tip (327) generally results in reduced force to penetrate relative to piercing tip (54). Generally, only blade (51) of piercing tip (54) only results in cutting of tissue. As a consequence, a hoop stress builds within the tissue as it is stretched around the conically shaped portion of piercing tip (54). This hoop stress increases the amount of force required to push piercing tip (54) though additional tissue. By contrast, piercing tip (327) of the present example includes second cutting portion (340) occupying the same general space as the conically shaped portion of piercing tip (54). Although piercing tip (327) still generates hoop stress in tissue during tissue piercing, this hoop stress is reduced because second cutting portion (340) provides additional tissue severing as tissue is stretched to the lateral diameter defined by base portion (350).

In addition to the above, piercing tip (327) further reduces force to penetrate via outward extension distance (d1) defined by first longitudinal cutting edge (332) and second longitudinal cutting edge (334). In particular, as can be seen in FIG. 8A, blade (51) of piercing tip (54) is generally extends outwardly such that blade (51) terminates at an outward position that is flush with cannula (52). By contrast, first longitudinal cutting edge (332) and second longitudinal cutting edge (334) extend beyond cannula (212) to define outward extension distance (d1). Outward extension distance (d1) provides additional tissue severing beyond the diameter of cannula (212) that further reduces hoop stresses in tissue as piercing tip (327) penetrates through tissue. This reduces drag that would otherwise be imparted on cannula (212).

Like with piercing tip (227) described above, piercing tip (327) of the present example is generally constructed using the same process described above with respect to FIG. 6. For instance, piercing tip (327) is initially constructed using an MIM process as similarly described above. Next, cutting edges (332, 334, 344) are further defined by a grinding process as similarly described above to bring cutting edges (332, 334, 344) into certain predefined tolerances.

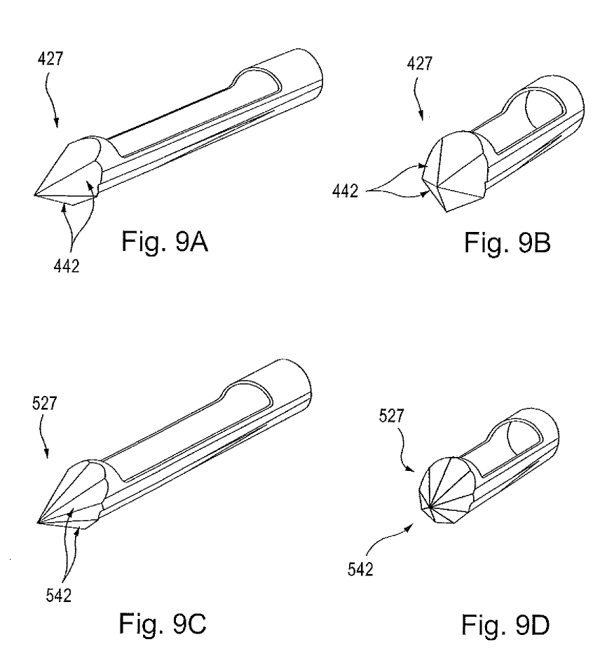

FIGS. 9A-9D show various alternative piercing tips (427, 527). Piercing tips (427, 527) are generally substantially the same as piercing tip (327) described above, except piercing tips (427, 527) include additional faceted surfaces (442, 542) beyond faceted surfaces (342) of piercing tip (327). For instance, piercing tip (427) shown in FIGS. 9A and 9B includes five facets. Alternatively, piercing tip (527) shown in FIGS. 9C and 9D includes nine facets. Of course, in other examples piercing tip (327) may be configured to include any suitable number of facets (342) as will be apparent to those of ordinary skill in the art in view of the teachings herein. It is believed, without intending to be bound thereby, that with an increase in the number of facets on the needle, that the “force-to-penetrate” the breast tissue will decrease which is a desirable feature of the needle(s) of the instant claimed invention.

While various alternative needles are described below as providing the above described features and functionality, it should be understood that other examples will be apparent to those of ordinary skill in the art in view of the teachings herein. It should be further understood that various features and/or structures of the needles described herein by be readily incorporated into other needles described herein.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1-20. (canceled)

21. A method of manufacturing a needle for use in a biopsy device, the method comprising:
(a) forming an insert member using a metal injection molding process, wherein the insert member defines a tube portion and a piercing tip;
(b) securing the insert member to an elongate cannula; and
(c) sharpening at least one cutting edge into a surface of the insert member defining the piercing tip.

22. The method of claim 21, wherein the step of forming the insert member further includes forming an insert member blank by solidifying a molten metal material within a mold, subjecting the insert member blank to a treatment process to remove residual binders or wax, and further subjecting the insert member blank to a sintering process, thereby forming the insert member.

23. The method of claim 22, wherein the step of forming the insert member further includes grinding of the insert member blank after subjecting the insert member blank to the sintering process.

24. The method of claim 21, wherein the step of securing the insert member within the elongate cannula further includes welding the insert member to the elongate cannula.

25. The method of claim 21, wherein the step of securing the insert member within the elongate cannula further includes laser welding the insert member to the elongate cannula.

26. A method of manufacturing a needle for use with a biopsy device, the method comprising:
(a) preparing a cannula by forming a notch in a distal end of the cannula;
(b) molding an insert member using a metal injection molding process, wherein the insert member defines a tube portion and a distal tip portion;
(c) aligning the insert member within the notch formed within the cannula; and
(d) securing the insert member to the cannula.

27. The method of claim 26, further comprising grinding one or more cutting portions into the distal tip portion defined by the insert member.

28. The method of claim 27, wherein the step of grinding one or more cutting edges into the distal tip portion includes grinding a first cutting portion and a second cutting portion, wherein the first cutting portion is ground to define a first cutting edge, wherein the second cutting portion is ground to define a second cutting edge.

29. The method of claim 26, further comprising subjecting the combination of the cannula and the insert member to a passivation process.

30. The method of claim 26, further comprising subjecting the insert member to a sintering process after the step of molding the insert member.

31. A needle for use with a biopsy device, wherein the needle comprises:
an elongate cannula extending from a body of the biopsy device; and
a single piece insert member defining a needle tip having a sharpened edge adapted to pierce tissue and a cutter shelf for receiving a cutter of the biopsy device, wherein the elongate cannula is configured to receive the insert member.

32. The needle of claim 31, wherein the insert member is a metal injection molded insert member.

33. The needle of claim 31, wherein the insert member is a metallic material configured for use with an injection molding process.

34. The needle of claim 31, wherein the elongate cannula defines a cutout portion, wherein the insert member is insertable into the cutout portion of the elongate cannula.

35. The needle of claim 31, wherein the needle tip of the single piece inset member includes a base portion, a first cutting portion, and a second cutting portion, wherein the first cutting portion and the second cutting portion extend distally from the base portion.

36. The needle of claim 31, wherein the elongate cannula defines a first diameter, wherein the needle tip of the single piece inset member includes a base portion, a first cutting portion, and a second cutting portion, wherein the first cutting portion and the second cutting portion extend distally from the base portion, wherein the base portion defines a first diameter, wherein the first diameter of the base portion corresponds to the first diameter of the elongate cannula.

37. The needle of claim 36, wherein the first cutting portion defines a first cutting edge and a second cutting edge, wherein at least a portion the first cutting edge and at least a portion of the second cutting edge extends outwardly beyond the first diameter of the base portion.

38. The needle of claim 36, wherein the second cutting portion defines a plurality of facets, wherein the first cutting portion is disposed between at least two of the plurality of facets of the second cutting portion.

39. The needle of claim 31, wherein the needle tip of the single piece inset member includes a base portion, a first cutting portion, and a second cutting portion, wherein the elongate cannula defines an external surface, wherein the base portion defines an external surface, wherein the external surface of the cannula and the external surface of the base portion are configured to align to form a smooth transition between the base portion and the cannula.

40. The needle of claim 31, wherein the insert member is laser welded to the cannula.
